(19) United States
(12) Patent Application Publication
(10) Pub. No.: US 2002/0168641 A1
(43) Pub. Date: Nov. 14, 2002

(54) Fluorescein-cyanine 5 as a fluorescence resonance energy transfer pair

(76) Inventors: Bruce Mortensen, Menlo Park, CA (US); Mary Chui, Cupertino, CA (US)

Correspondence Address: REED & ASSOCIATES, 800 MENLO AVENUE, SUITE 210, MENLO PARK, CA, 94025, US

(21) Appl. No.: 09/803,426

(22) Filed: Mar. 9, 2001

(51) Int. Cl. C12Q 1/68; G01N 33/53; G01N 33/537; G01N 33/543

(52) U.S. Cl. 435/6; 435/7.5; 435/7.92

(57) ABSTRACT

A method is provided for detecting the proximity of a first molecular segment to a second molecular segment. The method comprises covalently attaching fluorescein to a first molecular segment, covalently attaching cyanine 5 to a second molecular segment, and detecting the presence or absence of fluorescein-induced emission of cyanine 5 as a result of fluorescence resonance energy transfer when the first molecular segment and second molecular segment are in proximity to each other. Compositions and compounds comprising the fluorescein and cyanine 5 pair are also provided.

### TECHNICAL FIELD

The present invention relates generally to a novel fluorescence resonance energy transfer (FRET) pair; and more particularly relates to use of the pair in FRET-based assays.

### BACKGROUND

There is an increasing interest in the development and application of assays that do not require the use of radioactive isotopes. Although relying on radioactively tagged moieties offers ease of detection, these assays require special handling and safety precautions typically encountered when working with radioactive materials. In addition, the time between the preparation of the radiolabeled moiety to carrying out the assay is often a concern as the signal from radioactive isotopes decays over time. Furthermore, assays relying on the detection of radioactive moieties do not lend themselves to homogenous assay formats, i.e., assays that can be conducted using a “one-pot” procedure without the need of, for example, a washing step to remove unbound radioactive moieties. One type of assay that does not rely on radioactive isotopes and that can be used in a homogenous assay is the fluorescence resonance energy transfer assay.

FRET assays take advantage of a principle known as nonradiative energy transfer (radiationless energy transfer) to produce a signal. FRET assays are known as is the basic mechanism and theory behind these assays. See Szöllsi et al. (1998) Cytometry 34(4):159-179. Generally, a donor fluorophore is attached to one member of a potential binding pair, while an acceptor fluorophore is bound to the other member of the potential binding pair. The assay is allowed to incubate under binding conditions appropriate for the particular binding pair, and then is illuminated with light at the excitation wavelength of the donor. If the donor fluorophore and acceptor fluorophore are sufficiently close to each other, a change in the spectral response will occur. This change in the spectral response can be measured and indicates that donor and acceptor fluorophores are in close proximity to each other, thereby allowing one to conclude that the two members of the binding pair are, in fact, bound together. No change in the spectral response means there is an absence of binding as the donor fluorophore fluoresces normally, emitting light at its own characteristic wavelength.

The change that is detected in the spectral response may be one of several types. First, the acceptor fluorophore may absorb the energy given off from the donor fluorophore and retransmit the energy at its own unique frequency. Alternatively, the acceptor fluorophore may absorb the energy given off from the donor fluorophore and release the energy in another form, e.g., heat, thereby “quenching” the original energy given off from the donor fluorophore.

In all FRET assays, when the appropriate donor and acceptor fluorophores are located within a specific distance, known as the Forster's radius, energy from the donor is absorbed by the acceptor, which then emits at its own characteristic emission wavelength. The keys to any FRET assay is the choice of dyes to use and the configuration of the assay to ensure that the pair will either be located within the Forster's radius for energy transfer or not, which is directly dependent upon the occurrence of a certain event, e.g., binding.

The single best predictor for identifying potential dye pair candidates for use in FRET-based assays is to determine their spectral overlap. The “best” pairings have the emission peak of the donor overlapping with the excitation peak of the acceptor. See, for example, Schobel et al. (1999) Bioconjug. Chem. 10:1107-1114 and U.S. Pat. No. 6,150,097 to Tyagi et al.

Surprisingly, it has been found that although there is minimal, if any, spectral overlap for fluorescein and cyanine 5 (fluorescein excites at 495 nm and emits at 519, while cyanine 5 excites at 649 nm and emits at 670 nm), fluorescence resonance energy transfer nonetheless takes place when fluorescein and cyanine 5 are proximately located to each other.

A continuing need in this field is the desire for additional pairs of donor and acceptor fluorophores. As will be appreciated, the number of effective FRET pairs is limited, primarily because it has been understood that such pairs require a high degree of spectral overlap. Thus, additional pairs such as fluorescein and cyanine 5 allow for greater flexibility in assay design and the ability to distinguish even a greater number of signals in a multiplex assay, all within a homogenous assay format. The current invention addresses this and other needs of the art.

### SUMMARY OF THE INVENTION

Accordingly, it is a primary object of the invention to address the above-described need in the art by providing a method for detecting the proximity of a first molecular segment to a second molecular segment.

It is another object of the invention to provide such a method wherein fluorescein is covalently attached to a first molecular segment and cyanine 5 is covalently attached to a second molecular segment.

It is still a further object of the invention to provide such a method wherein the first molecular segment and second molecular segment are parts of the same molecule.

It is a yet another object of the invention to provide such a method wherein the first molecular segment and second molecular segment are parts of two distinct molecules that comprise a binding pair or are each directly or indirectly attached to a complementary member of a binding pair.

It is yet another object of the invention to provide such a method useful in a fluorescence resonance energy transfer-based assay.

It is a further object of the invention to provide such a method, wherein the fluorescence resonance energy transfer-based assay is a homogenous assay.

It is still a further object of the invention to provide such a method wherein the fluorescence resonance energy transfer based assay is a heterogeneous assay.

It is a further object of the invention to provide a composition comprising a first member of a binding pair directly or indirectly attached to flourescein and a second member of a binding pair directly or indirectly attached to cyanine 5, wherein the first and second members of the binding pair are associated so that the fluorescein and cyanine 5 are in fluorescence resonance energy transfer proximity to each other.

It is another object of the invention to provide such a composition wherein the first member of the binding pair is directly attached to fluorescein and the second member of the binding pair is directly attached to cyanine 5, and the direct attachment is effected through a covalent bond.

It is a further object of the invention to provide such a composition wherein the first member of the binding pair is indirectly attached to fluorescein and the second member of the binding pair is indirectly attached to cyanine 5, and the indirect attachment is effected through one or more linking moieties.

It is still another object of the invention to provide such composition wherein at least one member of the binding pair is indirectly attached to either fluorescein or cyanine 5, and the indirect attachment is effected through one or more linking moieties.

It is an additional object to provide a compound wherein a first molecular segment is covalently bound to fluorescein and a second molecular segment is bound to cyanine 5.

It is still a further object of the invention to provide a FRET-based assay having a dye pair, the improvement comprising using fluorescein and cyanine 5 as the dye pair.

Additional objects, advantages and novel features of the invention will be set forth in part in the description which follows, and in part will become apparent to those skilled in the art upon examination of the following, or may be learned by practice of the invention.

In one aspect of the invention then, a method is provided for detecting the molecular proximity between a first molecular segment and second molecular segment. The method involves (a) covalently attaching fluorescein to a first molecular segment, (b) covalently attaching cyanine 5 to a second molecular segment, and (c) detecting for the presence or absence of fluorescein-induced emission of cyanine 5 as a result of fluorescence resonance energy transfer when the first molecular segment and second molecular segment are in proximity to each other. The molecular segments may be a part of the same molecule. Alternatively, each molecular segment may independently be located on an individual member of two separate molecules, e.g., a binding pair, or be located on a linking moiety that is attached to another linking moiety or to a molecule of interest. The molecular segments are in proximity to each other when fluorescein-induced emission of cyanine 5 is detected because fluorescein and cyanine 5, each attached to a separate molecular segment, can only exhibit fluorescein-induced emission of cyanine 5 when the members of the dye pair are in proximity to each other, i.e., in “fluorescence resonance energy transfer” proximity to each other.

Preferably, the optimal proximity for resonance energy transfer between fluorescein and cyanine 5 is about 1 Å to about 100 Å, more preferably about 5 Å to about 80 Å, still more preferably about 10 Å to about 70 Å, and most preferably about 20 Å to 60 Å. Consequently, detection of fluorescein-induced emission of cyanine 5 indicates that the proximity of first molecular segment to the second molecular segment is about 1 Å to about 100 Å, more preferably about 5 Å to about 80 Å, still more preferably about 10 Å to about 70 Å, and most preferably about 20 Å to 60 Å. Thus, for example, when a ligand is coupled to fluorescein and the ligand's corresponding receptor is coupled to cyanine 5, fluorescein-induced emission of cyanine 5 occurs when the ligand and receptor are allowed to bind and the fluorescein and cyanine 5 are subsequently located within about 100 Å of each other.

As will be discussed in detail below, detecting cyanine 5 emission, covalently attaching the dyes, and so on may be accomplished using any art-known procedure. Furthermore, the method and dye pair can be used as a part of any FRET-based assay in any number of assay formats. It is preferred, however, that the assay is in the form of a homogenous assay.

In a related aspect of the invention, a composition is provided comprising a first member of a binding pair and a second member of a binding pair. The first member of the binding pair is indirectly or directly attached to fluorescein. The second member of the binding pair is also indirectly or directly attached to fluorescein. When a member of a binding pair is directly attached to a dye, the attachment is covalent. Indirect attachment results when one or more linker moieties, e.g., antibodies, antibody fragments, biotin molecules, etc., connect the dye to a molecule of interest, e.g., a member of a binding pair. Linker moieties, if present, must not separate the dye pair too far apart, since that would always prevent fluorescein-induced emission of cyanine 5. Thus, the first and second members of the binding pair are associated so that the fluorescein and cyanine 5 are in fluorescence resonance energy transfer proximity to each other.

In another aspect of the invention, a compound is provided comprising a first molecular segment covalently bound to fluorescein and a second molecular segment covalently bound to cyanine 5. Such compounds have many applications including assays that study conformational/enzymatic activity in addition to TaqMan® and “molecular beacon” assays, all of which are discussed infra.

### BRIEF DESCRIPTION OF THE DRAWINGS

FIG. 1 is a graph depicting the results of a FRET assay using 30 nM SA-FITC as donor and serial dilution of Cy5-bn as acceptor (unblocked versus blocked with excess biotin) according to the results in Example 1.

FIGS. 2A, 2B and 2C are graphs depicting the competitive binding curves of various ligands to the interleukin-1 receptor according to the results provided in Example 2.

FIGS. 3A, 3B, 3C and 3D are graphs depicting the competitive binding curves of various ligands to the interleukin-4 receptor according to the results provided in Example 3.

### DESCRIPTION OF THE INVENTION

Before describing the present invention in detail, it is to be understood that unless otherwise indicated this invention is not limited to particular molecular species, assay formats, or the like, as such may vary. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise. Thus, for example, reference to “a fluorescein-coupled moiety” includes a single fluorescein-coupled moiety and two or more fluorescein-coupled moieties, and “a cyanine 5-coupled moiety” refers to a single cyanine 5-coupled moiety or two or more cyanine 5-coupled moieties, and the like.

In this specification and in the claims that follow, the following terminology will be used in accordance with the definitions set forth below.

“Fluorescence resonance energy transfer” shall mean any non-radiative process whereby energy from a fluorescent donor molecule is transferred to an acceptor molecule, and the excitation of the fluorescent donor molecule enhances or changes the emission of the fluorescent acceptor molecule. As a consequence, the yield of the donor molecule's fluorescence emission is reduced. In resonance energy transfer, the efficiency of energy transfer is inversely correlated with the distance between the donor and acceptor molecules. Thus, the appearance of resonance energy transfer is a highly specific indicator of the proximity of the two molecules. Thus, a “fluorescence resonance energy transfer-based assay” is an assay based on the appearance or absence of resonance energy transfer between the molecules of the “dye pair.” Thus, the invention includes any FRET-based assay that incorporates use of fluorescein and cyanine 5 as a “dye pair.”

As used herein, the term “dye” is generic to both the acceptor molecule and the donor molecule used in an assay or method involving fluorescence resonance energy transfer. Unless the context clearly indicates otherwise, the dyes referred to herein are either fluorescein or cyanine 5. The term “dye pair” refers to the pairing of fluorescein and cyanine 5 for use in an assay or method involving fluorescence resonance energy transfer.

The term “peptide” refers to oligomers or polymers of any length wherein the constituent monomers are alpha amino acids linked through amide bonds, and encompasses amino acid dimers as well as polypeptides, peptide fragments, peptide analogs, naturally occurring proteins, mutated, variant or chemically modified proteins, fusion proteins, and the like. The amino acids of the peptide molecules may be any of the twenty conventional amino acids, stereoisomers (e.g., D-amino acids) of the conventional amino acids, or non-naturally occurring amino acids such as α,α-disubstituted amino acids, N-alkyl amino acids, β-alanine, naphthylalanine, 3-pyridylalanine, 4-hydroxyproline, O-phosphoserine, N-acetylserine, N-formylmethionine, 3-methylhistidine, 5-hydroxylysine, and nor-leucine. In addition, the term “peptide” encompasses peptides with posttranslational modifications such as glycosylations, acetylations, phosphorylations, and the like.

The term “oligonucleotide” is used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the oligonucleotide. More particularly, the term includes polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Ore., as Neugene) polymers, and other synthetic sequence-specific nucleic acid polymers, providing that the polymers contain nucleobases in a configuration that allows for base pairing and base stacking, such as is found in DNA and RNA. There is no intended distinction in length between the terms “polynucleotide,” “oligonucleotide,” “nucleic acid” and “nucleic acid molecule,” and these terms refer only to the primary structure of the molecule. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for, example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

The terms “coupled” and “attached” as used herein refers to attachment by covalent bonds or by non-covalent interactions (e.g., adsorption, physical immobilization, hydrophobic interactions, hydrogen bonds, affinity interaction, etc.). Covalent bonds may be, for example, ester, ether, phosphoester, amide, peptide, imide, carbon-sulfur bonds, carbon-phosphorus bonds, and the like. Dyes are coupled to molecular segments via a covalent bond. Methods for coupling dyes to molecular moieties are known in the art. Affinity interactions are non-covalent in nature and include, for example, the interaction between biotin and streptavidin, and an antibody and its corresponding antigen.

The term “binding pair” refers to first and second member molecules that specifically bind to each other. “Specific binding” of the first member of the binding pair to the second member of the binding pair is evidenced by the binding of the first member to the second member, or vice versa, with greater affinity and specificity than to other components in the sample. The binding between the members of the binding pair is typically noncovalent. Exemplary binding pairs include any haptenic or antigenic compound in combination with a corresponding antibody or binding portion or fragment thereof (e.g., digoxigenin and anti-digoxigenin; mouse immunoglobulin and goat anti-mouse immunoglobulin) and nonimmunological binding pairs (e.g., biotin-avidin, biotin-streptavidin, hormone [e.g., thyroxine and cortisol]-hormone binding protein, receptor-receptor agonist or antagonist (e.g., interleukin receptor-interleukin or an analog thereof), IgG-protein A, lectin-carbohydrate, enzyme-enzyme cofactor, enzyme-enzyme inhibitor, and complementary polynucleotide pairs capable of forming nucleic acid duplexes), and the like.

A “linking moiety,” when present, is a moiety linking the dye to a molecule of interest, e.g., one member of a binding pair. The dye will be covalently attached to a molecular segment located on the linking moiety, when present. The linking moiety, in turn, may be attached (either covalently or non-covalently) directly to the molecule of interest or indirectly to a molecule of interest through another linking moiety. Each linking moiety, when present, is independently selected from the group consisting of an antibody, antibody fragment, e.g., Fab, biotin and streptavidin. A biotinylated ligand non-covalently attached to dye-labeled streptavidin represents one example of indirect attachment of a linking moiety to one member of a binding pair.

“Molecular proximity” is defined as the distance between two molecular segments. “Fluorescence resonance energy transfer proximity” is a distance in which fluorescence resonance energy transfer between a dye pair can take place.

“Optional” or “optionally” means that the subsequently described circumstance may or may not occur, so that the description includes instances where the circumstance occurs and instances where it does not.

The Dye Pair:

As mentioned above, fluorescein and cyanine 5 exhibit resonance energy transfer when they are located sufficiently close to each other. This is so even though this pair does not exhibit significant, if any, spectral overlap. Thus, while it would not be expected for this particular dye pair, fluorescein and cyanine 5 exhibit resonance energy transfer.

Fluorescein is a small organic molecule and is well-known as a donor fluorophore. See, for example, Merck Index, 12^{th} Edition, 1996, Merck and Co., Rahway, N.J.

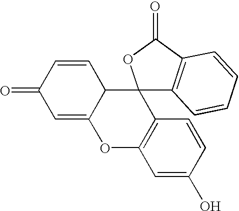

### Fluorescein

### 3′,6′-Dihydroxyspiro[isobenzofuran-1 (3H),9′-[9H]xanthen]-3-one

Fluorescein is available commercially from, for example, Sigma Corp., St. Louis, Mo. Alternatively, fluorescein may be produced synthetically by heating phthalic anhydride with resorcinol as described by Fischer (1922) J. Prakt. Chem. 104:123 and the Merck Index, cited supra.

Reactive versions of fluorescein for coupling purposes are readily available. Fluorescein isothiocyanate or “FITC” is a particularly preferred functionalized fluorescein and is available commercially (Sigma Corp., St. Louis, Mo.). Alternatively, FITC may be chemically synthesized by nitrating the fluorescein with NHO₃ and reducing the nitrate with nascent hydrogen produced by the addition of zinc and HCl. Adding thiophosgene then forms the isothiocyanate. Additional functionalized versions are known and are commercially available and include, for example, carboxyfluorescein succinimidyl ester and biotin-fluorescein.

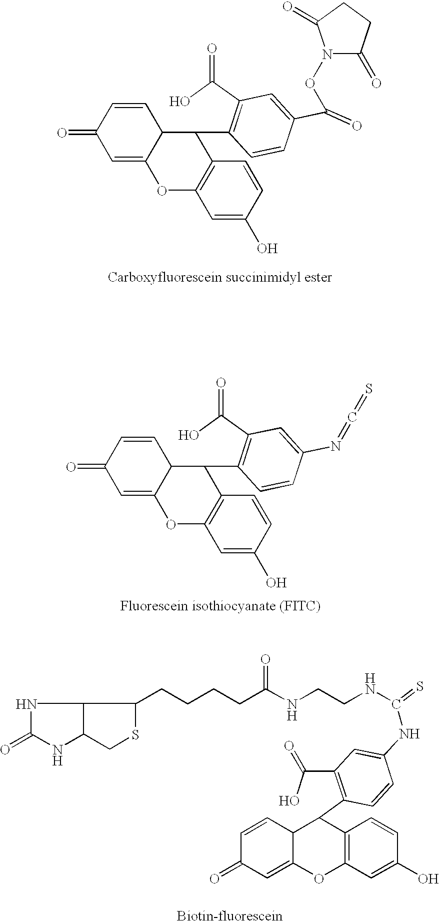

Cyanine 5 has the following structure:

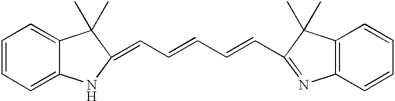

### Cyanine 5

### 2-[5-(3,3-Dimethyl-1,3-dihydro-indol-2-ylidene)-penta-1,3-dienyl]-3,3-dimethyl-3H-indole

Cyanine 5 is available in its reactive form. Reactive forms of cyanine 5 suitable for use in the present invention may have the structure of formula (I).

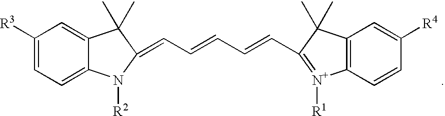

In formula (I), R¹ through R⁴ are reactive functional groups such as, for example, isothiocyanate, isocyanate, monochlorotriazine, carboxypentyl, dichlorotriazine, dichlorotriazinylamine, mono-or di-halogen substituted pyridine, maleimide, aziridine, sulfonyl halide, sulfate salt, acid halide, hydroxysuccinimide ester, hydroxysulfosuccinimide ester, imido ester, hydrazinyl, azidonitrophenyl, azide, 3-(2-pyridyldithio)-propionamide, amino, iodoacetamide, iodoacetamidomethyl, and the like. In addition, cyanine 5 may also be substituted with one or more straight or branched alkyl groups, e.g., a C₁₋₁₂ straight or branched alkyl group such as ethyl. Cyanine 5 and its reactive derivatives are available commercially. A particularly preferred cyanine 5 dye is Cy5™ from Amersham Pharmacia Biotech, Inc., Piscataway, N.J. Other reactive cyanine 5 dyes are also commercially available or may be synthetically produced as described in, for example, U.S. Pat. No. 5,268,486 to Waggoner et al. As illustrated in the structure of formula (I), characteristic of cyanine 5 is the 5-atom polymethine bridge, which is presumed to provide the dye's absorption and emission characteristics.

Coupling of A Dye to A Molecular Segment:

For use in the method described herein, the fluorescein and cyanine 5 must each be covalently attached to a particular molecular segment. The fluorescein and cyanine 5 may be covalently attached to molecular segments contained within a single molecule. For example, such an arrangement is useful for assays and experiments designed to elucidate the “active” conformation of a substrate for a corresponding receptor. Alternatively, the fluorescein and cyanine 5 may be covalently attached to molecular segments of separate molecules, e.g., one dye for each molecule in the binding pair. In this way, an assay designed to detect binding of the members of the binding pair is effected by detecting or measuring the fluorescein-induced emission of cyanine 5. Examples of particular assays and detection methods are described in detail below.

Covalently attaching the fluorescein or cyanine 5 dye to the desired molecular segment may be carried out using any conventional coupling procedure. As will be appreciated, coupling conditions, reagents and reactions will vary depending on the reactive group(s) present on the dye and/or the molecular moiety to be attached. Those of ordinary skill in the art can readily determine the conditions and reagents necessary to carry out the coupling reaction for the particular molecular moiety to be coupled. Procedures for coupling a dye to a molecular moiety are also described in the pertinent texts and literature, including, for example, March, Advanced Organic Chemistry: Reactions, Mechanisms, and Structure, 4^{th} Ed. (New York: John Wiley & Sons, 1992).

For molecules having an amine functionality, many reactive forms of the dyes are available that allow for the facile coupling of a dye to the amine functionality. One reactive form includes the isothiocyanate form of the dye, e.g., FITC. Covalent coupling is effected by combining the isothiocyanate form of the dye in an anhydrous polar organic solvent such as dimethylsulfoxide (DMSO) followed by addition of the amine-containing molecule. Generally, about 10 mg of the isothiocyanate can sufficiently label about 100 mg of a peptidic molecule containing an available amine. Covalent coupling can also be accomplished using a succinimidyl ester reactive form of the dye, e.g., cyanine 5 succinimidyl ester available from Amersham Pharmacia Biotech, Inc., Piscataway, N.J. (“Cy5-bis-OSY™” catalog no. PA15000) or synthetically produced following the procedure given in Mujumdar et al. (1993) Bioconj. Chem. 4(2)105-111, or the fluorescein N-hydroxy succinimidyl ester of carboxyfluorescein (available as FluorX™ from Research Organics, Inc., Cleveland, Ohio). Coupling is accomplished by adding the succinimidyl ester in an anhydrous organic polar solvent followed by addition of the amine-containing molecule. Usually, the dye is added to a solution containing the molecular segment. The molecular segment is generally added first, however, when the dye is in a lyophilized form that is subsequently solubilized in an aqueous solution. Examples of molecules and molecular segments that commonly have an amine functionality include, but are not limited to, proteins, polypeptides, antibodies, antibody fragments, receptors, ligands, enzymes, enzyme substrates, drugs, antigens, oligonucleotides, streptavidin, amine-containing sugars, e.g., glucosamine, galactosamine, and so on.

Oligonucleotides may also be covalently attached to fluorescein and/or cyanine 5. The oligonucleotide may be coupled to a dye using the procedures identified above with respect to amine-containing molecules and molecule segments by using, for example, a nonhybridized base such as a cytosine, guanine or thymine residue at, for example, one terminus of the oligonucleotide, or to a nonhybridized or hybridized modified base containing an amine functionality as described in Ganesh (1998) Curr. Sci. 75(12):1346-1354. Alternatively, fluorescein or cyanine 5-labeled nucleotides are commercially available as, for example, cyanine 5-deoxyuracil triphosphate or carboxyfluorescein-labeled deoxycytosine triphosphate (FluoroLink Cy5-dUTP™ product no. PA58021 and FluoroLink FluorX-dCTP™ product no. PA55022, respectively, available from Amersham Pharmacia Biotech, Inc., Piscataway, N.J.).

Labeled oligonucleotides can be prepared by, for example, nick translation. Briefly, labeled nucleotides along with the remaining three unlabeled nucleotides, the oligonucleotide (previously nicked with, for example, DNase I), and a polymerase are combined, mixed and allowed to incubate for at least 2 hours in a suitable buffer, e.g., a solution of 0.5M Tris-HCl (tris[hydroxymethyl]aminomethane-HCl), 0.1M MgSO₄ and 1 mM dithiothreitol. The resulting labeled oligonucleotide is allowed to precipitate by, for example, adding ammonium acetate in ethanol. The solution containing the precipitate is centrifuged, dried and resuspended in a storage buffer, e.g., a Tris-HCl/EDTA (ethylenediamine tetraacetic acid) solution, and stored until ready for use.

Additional coupling techniques are known. For example, coupling of fluorescein to bovine serum albumin (BSA), insulin, morphine, thyroxine and diphenylhydantoin is described in U.S. Pat. No. 3,998,943 to Ullman.

In some instances, a dye may be required to be coupled to a certain molecular segment on a particular molecule and not to others. For example, a molecule may have more than one amine group available for coupling, although coupling is desired only at one specific amine group located on the molecule. Techniques for coupling only to specific groups are well known in the art. For example, in the step-wise addition of individual monomers to a growing peptide or oligonucleotide chain, functionalized monomers bearing the dye can be incorporated into the growing polymer at a desired location. In addition, preferential coupling can be achieved by altering the reactivity of the dye, changing the ratio of dye to reactive groups, or changing the reactive groups.

For example, fusion proteins are often used in changing the ratio of dye to reactive groups to preferentially couple a dye. Briefly, a peptide such as an interleukin receptor antagonist having multiple primary amines on its surface can be fused to maltose binding protein using standard molecular biologic techniques. Because the maltose binding protein is much larger than the interleukin receptor antagonist, the majority of amines available for coupling will reside on the maltose binding protein rather than on the interleukin receptor antagonist. A limited amount of dye is then coupled to the fusion protein. As the dye will couple to the available amines in a random manner, the resulting conjugate will, for the most part, have the dye attached to the maltose binding protein rather than the interleukin receptor antagonist. Preferential coupling is particularly important when certain amines on a molecule are necessary for activity but are subject to being blocked with a dye.

In addition, preferential coupling may be effected by blocking certain reactive groups, coupling the dye, and then removing the blocking groups. For example, a protein that requires a free amino terminus for activity can be synthesized with a cysteine added at the end of a two glycine spacer at the carboxy terminus of the protein. Then, the amine reactive group of the dye can be modified such that is reacts with the free sulfllydryl group on the cysteine using techniques well known in the art. In this way, the N-terminus on the protein is free, thereby maintaining the protein's activity.

As will be appreciated by those of ordinary skill in the art, more than one fluorescein or cyanine 5 molecule may be covalently attached to one or more molecular segments. For peptidic molecules, preferably 1 to about 50, more preferably 1 to about 10, and most preferably about 1 to about 6 individual dye molecules will be covalently attached to the labeled molecule. The molar ratio between the fluorescein and cyanine 5 in the methods described herein is generally about equal, although ratios in the range of about 1:5 to about 5:1 of one dye to other may be used.

Fluorescence Resonance Energy Transfer-Based Assays:

The dye pairs can replace or be combined with other dye pairs in assays that rely on resonance energy transfer of a label. Such assays include, but are not limited to, affinity assays, detection assays and conformational/enzymatic activity assays.

Affinity Assays:

Often, knowing the affinity of a particular ligand, e.g., drug, peptide or substrate, for a particular receptor offers insight to the structure of the ligand or receptor (depending which is known) or is useful for screening purposes. Thus, for example, drug candidates are often screened for their ability to bind to a particular physiologic receptor, and those with the greatest affinity are chosen for further study. Advantageously, the present dye pair can be used in such assays that determine binding affinity.

Thus, for example, a known ligand for a particular receptor is labeled with, either directly or through one or more linking moieties, fluorescein and added under binding conditions to the corresponding receptor that has been labeled with cyanine 5. The resulting binding reaction between the labeled ligand and labeled receptor is allowed to equilibrate and the fluorescein-induced emission of cyanine 5 can be measured. Thereafter, a proposed ligand for the receptor is added and allowed to equilibrate. The fluorescein-induced emission is then detected a second time. If the fluorescein-induced emission remains unchanged, the proposed ligand has relatively less affinity for the receptor than for the known ligand. Alternatively, decreased emission indicates that the proposed ligand has a greater affinity for the receptor. Furthermore, the greater the decrease in the fluorescein-induced emission, the greater the affinity the proposed ligand has for the receptor.

As will be appreciated, this type of assay also allows for relative and quantitative determinations of particular ligands in solution by generating calibration curves. In addition, the ligand-receptor described above is not limited to any particular type of binding pair, and includes binding pairs such as, for example, antibody-protein, drug-receptor, protein-receptor, substrate-enzyme, etc. Furthermore, additional assay designs and formats for determining affinity are known and can be adopted for use with the present dye pair.

Detection assays:

The fluorescein and cyanine 5 dye pair can be used in a wide variety of detection assays. Detection assays are used to determine the presence or absence of a particular target molecule. By using an appropriate assay format and probe design, almost any type of target molecule, preferably a biological target molecule, can be detected. Thus, for example, the target molecule to be detected in the assay may be an oligonucleotide, including RNA, single-stranded DNA, double-stranded DNA, etc., peptides including small peptides, ligands for receptors, receptors, antibodies, antigens, etc.

In one such assay, the target oligonucleotide is placed in solution under hybridizing conditions with two dye-labeled oligonucleotide probes. One of the two dye-labeled oligonucleotide probes is coupled to fluorescein at its 5′ end while the other is coupled to cyanine 5 at its 3′ end. The sequences of the label probes are designed such that the two dyes are brought in close proximity to each other when each hybridizes to the target oligonucleotide. When such hybridization occurs, fluorescein-induced emission of cyanine 5 can be detected. For a general description of this type of assay, see Cardullo et al. (1988) P.N.A.S. 85:8790-8794 and Heller et al., EP 0 070 685.

Alternatively, a pair of oligonucleotide probes that are complementary to each other and to complementary strands of a target DNA molecule are labeled with the dye pair. Each probe has fluorescein coupled to its 3′ end and cyanine 5 coupled to its 5′ end. Under hybridizing conditions, and in the absence of the DNA target, the probes are hybridized and fluorescein-induced emission of cyanine 5 can be detected. If, however, the DNA target is present, the oligonucleotide probes will also hybridize to the target DNA, thereby decreasing the amount of fluorescein-induced emission of cyanine 5. For a general description of this type of assay, see Morrison et al. (1993) Biochemistry 32(12):3095-3104 and Morrison et al. EP 0 232 967.

In addition, strand displacement of a short oligonucleotide probe in favor of a larger target oligonucleotide can also enable detection of a target oligonucleotide. In this assay, a first probe comprises a non-nucleotidic moiety having fluorescein attached thereto in addition to an oligonucleotide region. A relatively short (i.e., shorter than the oligonucleotide target) second oligonucleotide probe having cyanine 5 coupled to one end is hybridized to the first probe such that both probes are in proximity to each other. Again, in the absence of the oligonucleotide target, the probes are hybridized and fluorescein-induced emission of cyanine 5 can be detected. When the target oligonucleotide is present, the shorter labeled probe will be replaced in favor of the longer oligonucleotide target, thereby decreasing the amount of fluorescein-induced emission of cyanine 5. For a general description of this type of assay, see U.S. Pat. No. 4,766,062 to Diamond et al.

Another assay is an assay based on an approach commonly referred to as “TaqMan®.” In this assay, a single-stranded probe oligonucleotide is labeled with both fluorescein and cyanine 5 such that the fluorescein and cyanine 5 are in optimal resonance energy transfer proximity to each other so that fluorescein-induced emission of cyanine 5 can occur and be detected. A DNA polymerase is then added that releases nucleotides, but only when the oligonucleotide is hybridized to the target strand. As the polymerase releases the nucleotides, the dye pair will be separated, thereby decreasing the amount of fluorescein-induced emission of cyanine 5. TaqMan® assays and variations thereof are described in U.S. Pat. No. 5,210,015 to Gelfand et al. and U.S. Pat. No. 5,538,848 to Livak et al.

Another type of detection assay uses an oligonucleotide probe referred to as a “molecular beacon.” In such an assay, the terminal ends of the oligonucleotide probe are complementary to each other with one end coupled to fluorescein and the other coupled to cyanine 5. Although the dye pair is held close to each other due to hybridization at each end of the oligonucleotide, a middle section of the oligonucleotide remains unhybridized. The result is an oligonucleotide forming a looped structure. The middle section of the oligonucleotide is complementary to the target oligonucleotide. In the absence of the target oligonucleotide, the probe remains in its looped structure and fluorescein-induced emission of cyanine 5 can be detected. When the oligonucleotide target is present, however, the oligonucleotide probe loop structure is broken in favor of hybridization to the target. When this occurs, the dyes of the dye pair are no longer in proximity to each other and the amount of fluorescein-induced emission of cyanine 5 is reduced or eliminated, indicating the presence of the target. This type of assay is described in U.S. Pat. No. 5,925,517 to Tyagi and in Tyagi et al. (1996) Nature Biotechnology 14:303-308.

Conformational/Enzymatic Activity Assays:

Understanding the conformational structures of macromolecules or complex binding pairs such as receptors and ligands offers the ability to better understand physiological and biological systems. Use of a dye pair such as fluorescein and cyanine 5 offers the ability to measure distances in the range of 0.5 to 10 nm (5 Å to 100 Å), i.e., a distance within the Forster's radius for the present dye pair.

For example, it is known that the physiological activity of a protein is at least partially influenced by its conformation or three-dimensional structure. Depending on the protein, the conformation may change, resulting in a change of activity. Knowing the “active” conformation provides useful information concerning the structure-activity relationship (SAR) for that particular protein. In operation, fluorescein is coupled to one region of protein while cyanine is coupled to a second region. If the protein is in an “extended” conformation (e.g., more linear), there will be no fluorescein-induced emission of cyanine 5. If, however, the protein folds though enzymatic activity or to bind to a receptor, the members of the dye pair may be placed in proximity to each other and fluorescein-induced emission of cyanine 5 can occur and be detected. Such assays have been performed on oligosaccharides using other resonance-energy transfer pairs. See, for example, Lee (1997) J. Biochem. 121:818-825, Rice et al. (1991) Biochemistry 30:6646-6655, and Lee (1992) FASEB J. 6:3193-3200.

Also, the present dye pair can be used to monitor certain enzymatic reactions. For example, the fluorescein and cyanine 5 dyes can be coupled in proximity to each other on the same molecule, thereby allowing for the detection of fluorescein-induced emission of cyanine 5. Upon enzymatic degradation of the molecule, the molecule may be cleaved such that the dyes are no longer in proximity to each other. By perpetually monitoring the decrease in cyanine 5 emission, the progress of the reaction can be monitored in real time. Assays such as these are known for use with different dye pairs and are described at, for example, Lee (1997), supra, Matsuoka et al. (1994) Tetrahedron: Assymetry 5:2335-2338 and Lee et al. (1995) Anal. Biochem. 230:31-36.

As will be appreciated by those skilled in the art, the above assays can be homogenous assays or heterogeneous assays. In homogenous assays, no separate separation step is required as the dye pair is able to send two different signals: one when the two dyes are in fluorescence resonance energy transfer-proximity and another when they are not. The advantage of homogenous assays is that they require fewer steps, which can simplify the procedure and reduce cost and wasted materials. The assay may be heterogeneous as well. For example, separation of a mixture of unbound dye-labeled probes from bound probe may be useful in reducing background.

Other assays for which the present dye pair are suited will be readily apparent to those of ordinary skill in the art. Such assays are described in, for example, Bagwell, EP 0 601 889.

Detection Methods:

Fluorescein-induced emission of cyanine 5 can be determined using any suitable technique known to those skilled in the art. The detection method generally employs a light source that illuminates a mixture containing the labeled material, i.e., the molecule segments covalently attached to a dye. Then, a detection apparatus is used to detect fluorescent energy that is emitted by the mixture. Preferred detection devices include fluorescence spectrometers, absorption spectrophotometers, fluorescence microscopes, transmission light microscopes, transmission light flow cytometers and fiber optic sensors.

The light used to illuminate the mixture containing the dye pair will emit fluorescent light having a wavelength between from about 440 nm to about 540 nm, preferably from about 465 nm to about 515 nm, an most preferably about 485 nm. Light at these wavelengths are absorbed by fluorescein. For example, commercially available Argon lasers are suitable light sources as they emit light having a specific wavelength suitable for absorbance by fluorescein. Alternatively, or in addition to a light source emitting a specific wavelength, filters may be used with lamps, e.g., Xenon lamps, which emit a broad range of wavelengths. The filter is preferably a 485 nm peak excitation filter so that light having an undesirable wavelength is eliminated or reduced. When the dye pair is in resonance energy-exchange proximity to each other, the cyanine 5 emission is read using a detection device. Preferably the emission is measured in order to quantify the signal. Filters may also be used when reading the emission, with for example, a 682 emission filter. The filter is used to eliminate interference from other wavelengths when detecting the emitted light.

Molecular Segments Used in the Assay:

The dyes used in the method are coupled to various molecules or moieties through a functional group or other reactive site, e.g., amine, located within the molecule or moiety. As previously indicated, each dye is attached covalently to a molecular segment. Each molecular segment may be a part of a single molecule or moiety. Alternatively, each molecular segment may individually be associated with one particular member of a binding pair. For example, each dye may be covalently linked to a separate member of a binding pair. In the schematics that follow, the binding pair is a receptor-ligand binding pair.

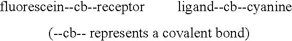

In addition, one dye may be covalently coupled to one member of a binding pair, while the second dye may be covalently bound to a linking moiety that is covalently or non-covalently bound to a member of a binding pair. The following two schematics show fluorescein covalently bound to an antibody serving as a linking moiety that, in turn, is non-covalently bound a receptor.

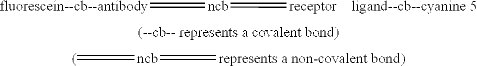

Also, both dyes may be covalently bound to one or more linking moieties that are covalently or non-covalently bound to a member of a binding pair. In the schematic immediately below, cyanine 5 is covalently bound to streptavidin (a linking moiety), which is non-covalently bound to biotin (another linking moiety), which is covalently bound to a ligand.

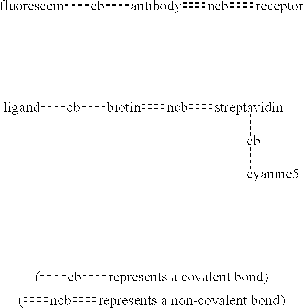

Of course, other arrangements are possible and may include more than one molecular segment. For example, the total number of molecular segments may be from 2 to about 12, more preferably from 2 to about 8. Any given molecule may include more than a single molecular segment for binding a dye.

The ultimate arrangement of the dye pair, however, must allow for fluorescein-induced emission of cyanine 5 at one state, e.g., when the members of a binding pair are bound to each other, and the absence of fluorescein-induced emission of cyanine 5 at a different state, e.g., when the the members of the binding pair are not bound to each other. Thus, for example, linking moieties, when present, must not separate the dye pair to such an extent that fluorescein-induced emission of cyanine 5 would not occur even when the binding pair are bound to each other.

Although the molecule, e.g., a member of the binding pair, a linking moiety, etc., is not limited with respect to type, the molecule is preferably an oligonucleotide or a peptide, e.g., an antibody, protein, receptor, etc. As will be appreciated, many proteins and oligonucleotides can be isolated from samples obtained in nature and purified using standard techniques, such as chromatography. In addition, many of the molecules for use in the method are commercially available from, for example, Sigma, Corp., St. Louis, Mo. Alternatively, the desired molecule may be synthesized. Techniques of synthesizing proteins, oligonucleotides, etc. are well known in the art.

For proteins, peptides and polypeptides, standard solid phase peptide synthesis techniques are preferred. Such techniques are described, for example, by Merrifield (1963) J. Am. Chem. Soc. 85:2149.

The oligonucleotides used in the method may be prepared by oligonucleotide synthesis or by recombinant methods. Typically, methods for synthesizing oligonucleotides involve sequential addition of 3′-blocked and 5′-blocked nucleotide monomers to the terminal 5′-hydroxyl group of a growing oligonucleotide chain. Each addition of a blocked nucleotide is effected by nucleophilic attack of the terminal 5′-hydroxyl group of the growing chain on the 3′-position of the added monomer, which is typically a phosphorus derivative such as a phosphotriester, phosphoramidite, or the like. Such methodology will be known to those skilled in the art and is described in the pertinent texts and literature, e.g., in D. M. Matteuci et al. (1980) Tet. Lett. 521:719, U.S. Pat. No. 4,500,707 to Caruthers et al., and U.S. Pat. Nos. 5,436,327 and 5,700,637 to Southern et al.

Oligonucleotide probe sequences are determined using standard techniques known in the art, and are can be derived from the sequence of the target oligonucleotide. The sequence for any given target oligonucleotide can be determined experimentally or obtained by accessing available databases such as the GenBank® database (National Center for Biotechnology Information, Bethesda Md.).

The antibodies for use in the method can be obtained by injecting into an animal, e.g., a rabbit, an antigen, e.g., a foreign protein, optionally coupled to an immunogenic moiety. The immunogenic moiety may be, for example, KLH (keyhole limpet hemocyanin), diphtheria toxoid or bovine serum albumen, with KLH preferred. The immunogenic moiety is coupled to the antigen using conventional coupling reagents such as glutaraldehyde, m-maleimidobenzoyl-N-hydroxysuccinimide ester, carbodiimide or bis-biazotized benzidine. The use of the immunogenic moiety increases the yield of the antibody that is ultimately collected from the animal. The antigen that is injected into the animal may be synthetically produced as described in above, or may be obtained from natural sources (if available). After a sufficient time, serum is collected from the animal and the antibodies are collected via any art-known method, e.g., centrifugation.

Fluorescein and cyanine 5 accordingly represent a pair of dye molecules that can be used in any resonance energy-transfer based assay. The pair is useful in affinity assays, detection assays, and assays designed to better understand conformational and enzymatic systems. Each dye can be coupled to a variety of molecule types, thereby allowing for use in assays that incorporate proteins, oligonucleotides, and other molecules as well.

In addition, fluorescein and cyanine 5, like other conventional FRET pairs, can be used to conduct solution-phase or homogenous assays. In such assays, washing steps or separation steps are rendered obsolete as the dye pair allows for signal differentiation based on the proximity of the dye pair to each other. So long as the assay is designed correctly, i.e., the result that is tested for has a nexus to the proximity of the dye pair, fluorescein and cyanine 5 are effective for use in homogenous assays. As will be readily appreciated by those skilled in the art, homogenous assays offer the advantages of decreasing expense and minimizing complexity as less handling is required. Furthermore, the dye pair is safe and does not require the special precautions taken when using radioactive labels.

Fluorescein and cyanine 5 represent a dye pair that is especially useful due to the large spectral separation between the two dyes. The large spectral separation allows for fluorescent contributions from the donor (fluorescein) to be easily filtered out. Thus, fluorescein can be excited using a 485 nm peak excitation filter, which will not excite cyanine 5. Moreover, the cyanine 5 emission can be read with a 682 nm peak emission filter, which receives no contribution from either the excitation source or fluorescein emission. Thus, data can be plotted without the need to calibrate contribution ratios for the respective dyes.

The addition of a unique dye pair, also allows for the creation of additional multiplex assays, all within a “one pot” procedure. That is, fluorescein and cyanine 5 represent another resonance energy transfer pair that can be used along with other pairs in multiplex assays. Thus, the number of possible assays conducted in a single multiplex assay is increased. Increasing the number of tests performed in a single assay is economical and more efficient than conducting separate, individual assays.

It is to be understood that while the invention has been described in conjunction with the preferred specific embodiments thereof, the foregoing description, as well as the examples that follow, are intended to illustrate and not limit the scope of the invention. Other aspects, advantages and modifications will be apparent to those skilled in the art to which the invention pertains. All patents, patent applications, journal articles and other references cited herein are incorporated by reference in their entireties.

In the following examples, efforts have been made to ensure accuracy with respect to numbers used (e.g., amounts, temperature, etc.) but some experimental error and deviation should be accounted for. Unless indicated otherwise, temperature is in degrees C and pressure is at or near atmospheric. All components were obtained commercially, e.g., from Molecular Probes, Inc (Eugene, OR), Sigma Corp. (St. Louis, Mo.), etc., unless otherwise indicated.

### EXAMPLES

The practice of the present invention will employ, unless otherwise indicated, conventional techniques of organic chemistry, and the like, which are within the skill of the art. Such techniques are explained fully in the literature.

The following examples are put forth so as to provide those of ordinary skill in the art with a complete disclosure and description of how to make and use the invention, and are not intended to limit the scope of what the inventors regard as their invention.

### Example 1

Biotin was labeled with Cy5™ (Amersham Pharmacia Biotech, Inc., Piscataway, N.J.) and streptavidin was labeled with FITC. The streptavidin-labeled fluorescein (SA-FITC) was separately added to 30 nM of unblocked Cy5-labled biotin (Cy5-bn) and 30 nM of “blocked” biotin. “Blocked” biotin was obtained by adding excess biotin to the reaction mixture. The procedure was repeated using dilutions of 10 nM Cy5-bn and 3 nM Cy5-bn.

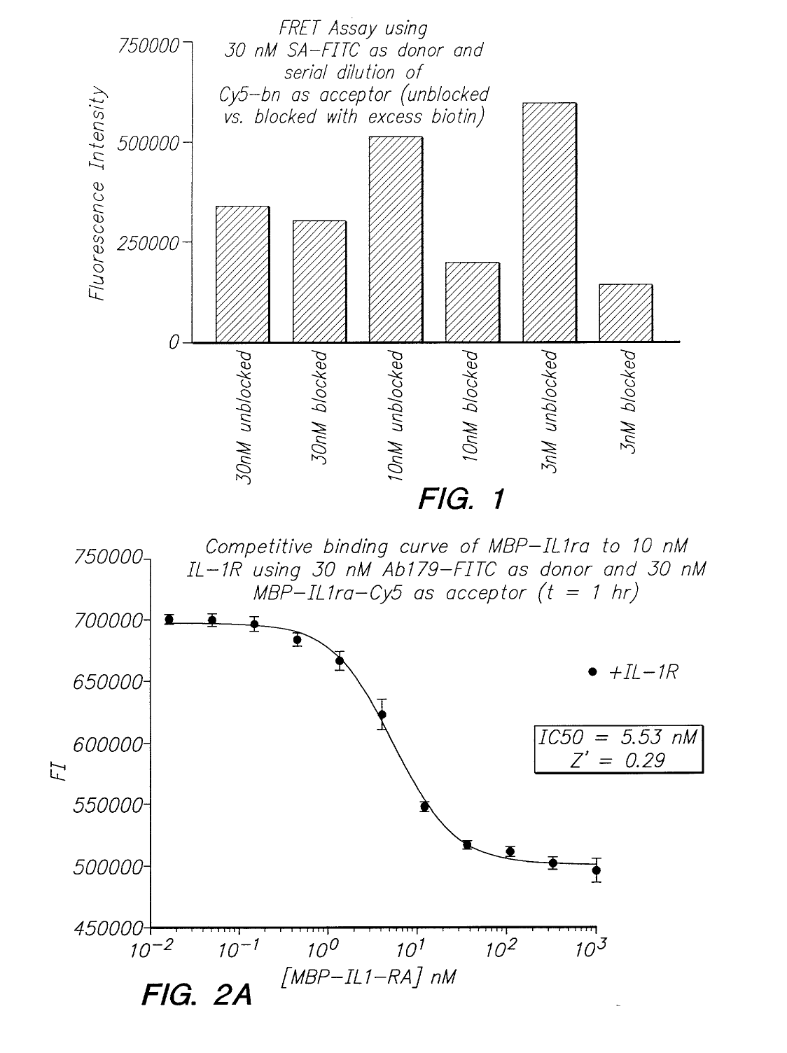

Fluorescence intensity was measured using LJL Analyst AD Assay Detection System (LJL Biosystems, Inc., Sunnyvale, Calif.) in fluorescence intensity mode using a Xenon lamp that was set on continuous excitation. No attenuator was used. The apparatus used a 485-20 nm excitation filter, a 670 nm dichroic mirror, and a 682-22 nm emission filter. One read per well was performed using 100,000 μsec integration time. The apparatus was run with LJL Biosystem's CriterionHost v2.00.11 software and instrument control program version 2.09. All filters were obtained by LJL Biosystems. The data were analyzed and displayed graphically using either Microsoft Excel 97 (Microsoft Corp.) or GraphPad Prism v3.02 (GraphPad Software). The results are provided in FIG. 1.

The experiment demonstrated that Cy5™ and fluorescein exhibited resonance energy transfer.

### Example 2

A competitive binding assay was performed using an interleukin-1 antagonist (AF11733) and the natural ligand, interleukin-1 (IL1). See Martins et al. (1999) Anal. Biochem. 273:20-31 for a characterization of AF11733. The reagents for this assay included the interleukin-1 receptor (IL-1R) a transmembrane receptor, antibody-labeled FITC (Ab179-FITC), maltose-binding protein-ILI-RA-Cy5 (MBP-IL1-RA-Cy5), and AF11733 at 1:1000 dilution. The assay volume was as follows: 2 μL of compound/inhibitor, 4 μL of IL-1R, 2 μL of MBP-IL1-RA-Cy5 or AF11733-Cy5, and 2 μL of Ab179-FITC. Assays were performed in plates containing 384 wells (OptiPlate™ available from Packard Instrument Co., Meriden, Conn., product number 6005256) The Ab179-FITC binds to the receptor so that the fluorescein dye may be in proximity to a Cy5-labeled ligand. The fluorescence intensity (FI) was read as described in Example 1.

The following chart describes the assay conditions and provides the experimentally obtained inhibitory 50 (IC50) values.

| | | MBP-IL-RA | AF11733 |
|---|---|---|---|
| Donor | [Ab179-FITC] | 30 nM | 60 nM |
| Acceptor | [Peptide-Cy5] | 30 nM | 1:1000 |
| IC50: | After 1 hour | 5.53 nM | 7.13 nM |
| | Overnight | 6.19 nM | |

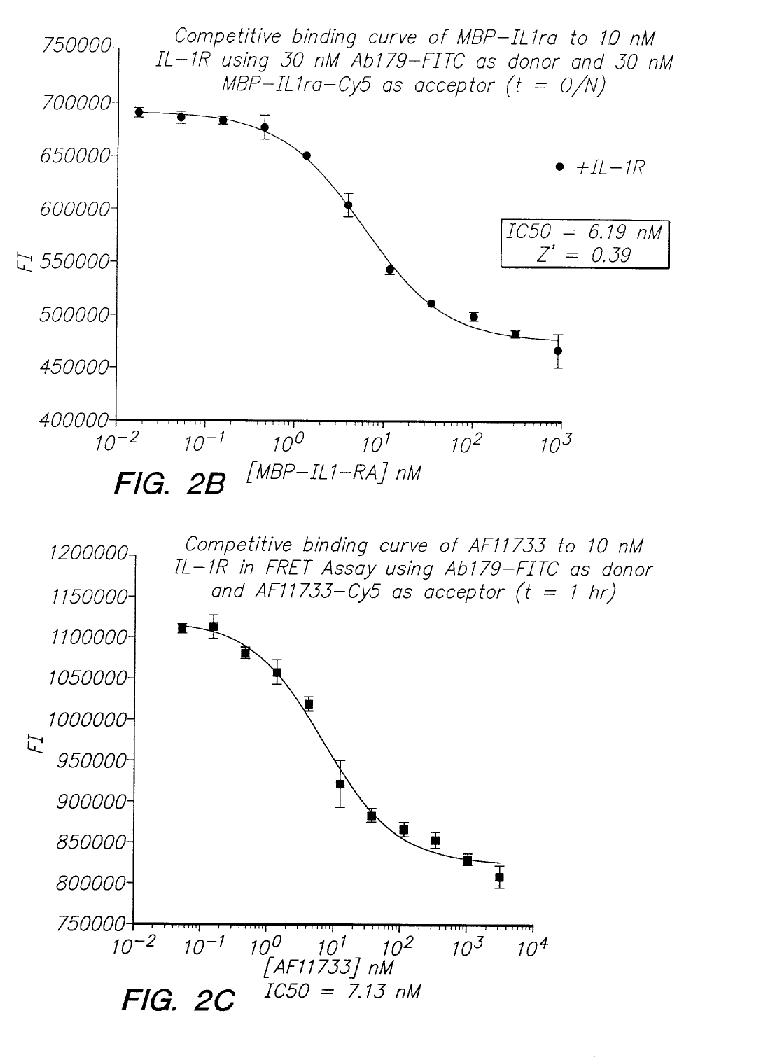

The competitive binding curves of MBP-IL-RA after 1 hour and following overnight incubation are presented in FIG. 2A and FIG. 2B, respectively. The competitive binding curve of AF11733 after 1 hour is presented in FIG. 2C.

This experiment demonstrated that the dye pair is effective for use in FRET assays that determine relative affinity.

### Example 3

A competitive binding assay was performed using a modified interleukin-4 agonist (IL4 His 6). IL4 His 6 consists of the native IL4 sequence to which a BirA recognition sequence followed by six histidine residues. The six histidine residues function as an affinity tag and allow the protein to be purified on a Ni-chelation column (Qiagen Inc., Valencia, Calif., product number 969263). Varying concentrations of IL4 His 6 were added following the procedure of Example 2.

The following chart describes the assay conditions and provides the experimentally obtained inhibitory 50 (IC50) values.

| | | 1:100 IL-4A | 1:10 IL-4R | 1:10 IL-4R |
|---|---|---|---|---|
| Donor | [Ab179-FITC] | 60 nM | 30 nM | 60 nm |
| Acceptor | [IL4-His6-Cy5] | 1:100 | 1:50 | 1:50 |
| IC50: | After 1 hour | 26.67 nM | 122.8 nM | 132.3 nM |
| | Overnight | 13.45 nM | | |

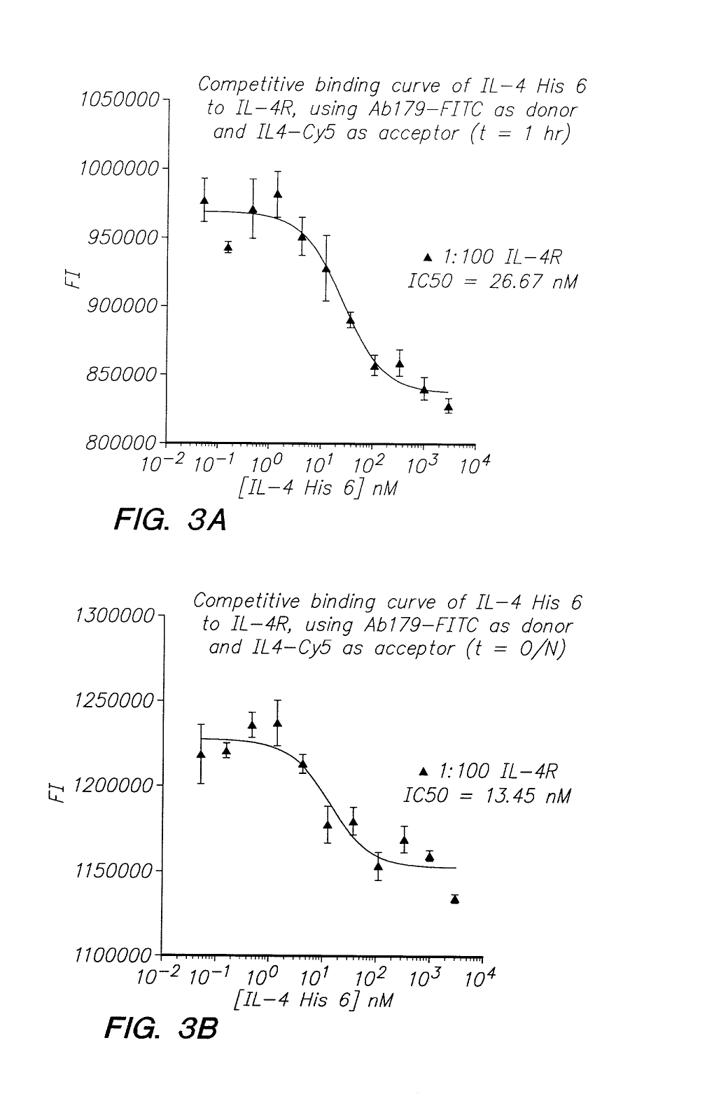

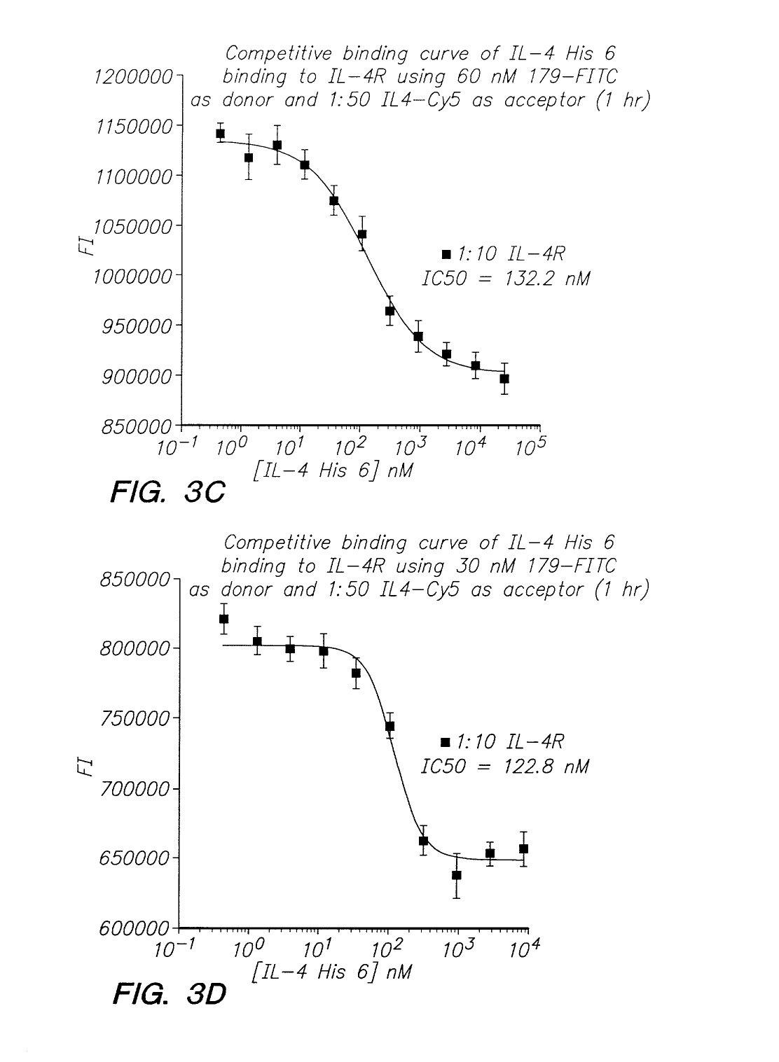

The competitive binding curves of 1:100 IL-4R after 1 hour and following overnight incubation are presented in FIG. 3A and FIG. 3B, respectively. The competitive binding curve of 1:10 IL-4R with concentrations of Ab179-FITC of 30 nM and 60 nM after 1 hour are presented in FIG. 3C and FIG. 3D, respectively.

This experiment also demonstrated that the dye pair is effective for use in FRET assays that determine relative affinity.

We claim:

1. A method for detecting the proximity of a first molecular segment to a second molecular segment, comprising:
a) covalently attaching fluorescein to a first molecular segment;
b) covalently attaching cyanine 5 to a second molecular segment; and
c) detecting the presence or absence of fluorescein-induced emission of cyanine 5 as a result of fluorescence resonance energy transfer when the first molecular segment and second molecular segment are in proximity to each other.

2. The method of claim 1, wherein the method is used as part of a fluorescence resonance energy transfer-based assay.

3. The method of claim 2, wherein the resonance energy transfer-based assay is a heterogeneous assay.

4. The method of claim 2, wherein the resonance energy transfer assay is a homogenous assay.

5. The method of claim 1, wherein the first molecular segment and second molecular segment are parts of the same molecule.

6. The method of claim 1, wherein the first molecular segment and second molecular segment are not parts of the same molecule.

7. The method of claim 6, wherein the first molecular segment is a part of a first member of a binding pair or a part of a first linker moiety that is directly or indirectly attached to the first member of the binding pair, and the second molecular segment is a part of a second member of a binding pair or a part of a second linker moiety that is directly or indirectly attached to the second member of the binding pair.

8. The method of claim 7, wherein the first molecular segment is a part of the first member of the binding pair, and the second molecular segment is a part of the second member of the binding pair.

9. The method of claim 7, wherein the first molecular segment is a part of a first linker moiety that is directly or indirectly attached to the first member of the binding pair, and the second molecular segment is a part of a second linker moiety that is directly or indirectly attached to the second member of the binding pair.

10. The method of claim 9, wherein the first or second linker moiety is independently selected from the group consisting of an antibody, antibody fragment, biotin and streptavidin.

11. The method of claim 7, wherein the binding pair comprises an enzyme-enzyme substrate pair.

12. The method of claim 7, wherein the binding pair comprises an antibody-antigen pair.

13. The method of claim 7, wherein the binding pair comprises a biological receptor-ligand pair.

14. The method of claim 7, wherein the binding pair comprises complementary oligonucleotides.

15. The method of claim 1, wherein the proximity is about 1 Å to about 100 Å.

16. The method of claim 15, wherein the proximity is about 5 Å to about 80 Å.

17. The method of claim 16, wherein the proximity is about 10 Å to about 70 Å.

18. The method of claim 17, wherein the proximity is about 20 Å to about 60 Å.

19. The method of claim 1, wherein the fluorescein-induced emission of cyanine 5 is detected using a 682 peak emission filter.

20. The method of claim 1, wherein the total number of first and second molecular segments is from 2 to about 12.

21. The method of claim 20, wherein the total number of first and second molecular segments is from 2 to about 8.

22. The method of claim 2, wherein the fluorescence resonance energy transfer-based assay is used to determine affinity of ligand to a receptor.

23. The method of claim 2, wherein the fluorescence resonance energy transfer-based assay is used to detect a target molecule selected from the group consisting of a protein, an RNA, a DNA, an antigen and an antibody.

24. The method of claim 2, wherein the resonance energy transfer-based assay is used to determine the structural conformation of a molecule.

25. The method of claim 2, wherein the resonance energy transfer-based assay is used to monitor enzymatic reactions.

26. A composition comprising a first member of a binding pair directly or indirectly attached to fluorescein and a second member of the binding pair directly or indirectly attached to cyanine 5, wherein the first and second members of the binding pair are associated so that the fluorescein and cyanine 5 are in fluorescence resonance energy transfer proximity to each other.

27. The composition of claim 26, wherein the first member of the binding pair is directly attached to fluorescein and the second member of the binding pair is directly attached to cyanine 5, and the direct attachment is effected through a covalent bond.

28. The composition of claim 26, wherein the first member of the binding pair is indirectly attached to fluorescein and the second member of the binding pair is indirectly attached to cyanine 5, and the indirect attachment is effected through one or more linking moieties.

29. The composition of claim 26, wherein at least one member of the binding pair is indirectly attached to either fluorescein or cyanine 5, and the indirect attachment is effected through one or more linking moieties.

30. The composition of claim 26, wherein the binding pair comprises an enzyme-enzyme substrate pair.

31. The composition of claim 26, wherein the binding pair comprises an antibody-antigen pair.

32. The composition of claim 26, wherein the binding pair comprises a biological receptor-ligand pair.

33. The composition of claim 26, wherein the binding pair comprises complementary oligonucleotides.

34. The composition of claim 26, wherein the fluorescence resonance energy transfer proximity is about 1 Å to about 100 Å.

35. The composition of claim 34, wherein the fluorescence resonance energy transfer proximity is between about 5 Å to about 80 Å.

36. The composition of claim 35, wherein the fluorescence resonance energy transfer proximity is between about 10 Å to about 70 Å.

37. The composition of claim 36, wherein the fluorescence resonance energy transfer proximity is between about 20 Å to about 60 Å.

38. A compound comprising a first molecular segment covalently bound to fluorescein and a second molecular segment covalently bound to cyanine 5.

39. The compound of claim 38, wherein the fluorescein and cyanine 5 are in fluorescence resonance energy transfer proximity to each other.

40. The compound of claim 39, wherein the fluorescence resonance energy transfer proximity is about 1 Å to about 100 Å.

41. The compound of claim 40, wherein the fluorescence resonance energy transfer proximity is between about 5 Å to about 80 Å.

42. The compound of claim 41, wherein the fluorescence resonance energy transfer proximity is between about 10 Å to about 70 Å.

43. The compound of claim 42, wherein the fluorescence resonance energy transfer proximity is between about 20 Å to about 60 Å.

44. In a FRET-based assay having a dye pair, the improvement comprising using fluorescein and cyanine 5 as the dye pair.
